(19) United States
(12) Patent Application Publication
(10) Pub. No.: US 2026/0198963 A1
(43) Pub. Date: Jul. 16, 2026

(54) TROCAR ADAPTER AND SURGICAL ROBOT INCLUDING THE SAME

(71) Applicant: LIVSMED INC., Seongnam-si (KR)

(72) Inventors: Jae Hoon SHIN, Gwangju-si (KR); Dong Hyuk KIM, Seoul (KR); Se Ra KIM, Suwon-si (KR); Young Wan KIM, Seongnam-si (KR)

(21) Appl. No.: 19/445,994

(22) Filed: Jan. 12, 2026

(30) Foreign Application Priority Data
Jan. 13, 2025 (KR) 10-2025-0005052

(51) Int. Cl. A61B 17/34; A61B 34/30; A61B 46/10; A61B 17/00

(52) U.S. Cl. CPC A61B 17/3423; A61B 34/30; A61B 46/10; A61B 2017/00486; A61B 2034/302

(57) ABSTRACT

Provided is a surgical robot including a trocar body into which a surgical instrument is inserted, a robotic arm in which the trocar body is arranged, and a trocar adapter that connects the trocar body to the robotic arm, wherein the trocar adapter includes a fastening body mounted on the robotic arm and to which the trocar body is coupled, a cover portion arranged between the trocar body and the fastening body and coupled to one side of the fastening body, and a drape portion that is arranged between the fastening body and the cover portion, and surrounds at least a portion of the robotic arm.

### CROSS-REFERENCE TO RELATED APPLICATION

This application is based on and claims priority under 35 U.S.C. §119 to Korean Patent Application No. 10-2025-0005052, filed on January 13, 2025, in the Ministry of Intellectual Property (MOIP) of the Republic of Korea, the disclosure of which is incorporated by reference herein in its entirety.

### BACKGROUND

### 1. Field

The present disclosure relates to a trocar adapter and a surgical robot including the same, and more particularly, to a surgical robot for use in laparoscopic surgery or various other surgeries, and a trocar adapter mounted on the surgical robot.

### 2. Description of the Related Art

In medical terms, surgery refers to the treatment of disease by cutting, incising, or otherwise manipulating the skin, mucous membranes, or other tissues by using medical devices. In particular, open surgery for incising and opening the skin of a surgical site to treat, shape, or remove an organ or the like therein causes issues such as bleeding, side effects, patient's pain, or scarring. Therefore, recently, surgery performed by forming a certain hole on a skin and inserting only a medical device, for example, a laparoscopic instrument or a surgical instrument, or surgery using a robot has been spotlighted as an alternative.

A surgical instrument refers to a tool for operating on a surgical site by a doctor manipulating an end tool provided at one end of a shaft passing through a hole drilled in the skin with a certain driving part or a robotic arm. The end tool provided in the surgical instrument performs a rotation motion, a gripping motion, a cutting motion, and the like, through a certain structure.

This type of laparoscopic surgery is a surgical method performed while observing the surgical site of the abdominal cavity, by forming a tube that accesses the patient's abdomen by using a surgical instrument called a trocar, and inserting a surgical instrument such as a laparoscope into the surgical site within the abdominal cavity through the trocar.

The above-mentioned background art is technical information possessed by the inventor for the derivation of the present disclosure or acquired during the derivation of the present disclosure, and cannot necessarily be said to be a known technique disclosed to the general public prior to the filing of the present disclosure.

### SUMMARY

The present disclosure provides a trocar adapter for a surgical robot for use in laparoscopic surgery or various other surgeries, which is capable of connecting a robotic arm to a trocar body into which a surgical instrument is inserted, while allowing for easy attachment and detachment of a sterile drape to and from the robotic arm, and a surgical robot including the trocar adapter.

According to an embodiment of the present disclosure, a surgical robot includes a trocar body into which a surgical instrument is inserted, a robotic arm in which the trocar body is arranged, and a trocar adapter that connects the trocar body to the robotic arm, wherein the trocar adapter includes a fastening body mounted on the robotic arm and to which the trocar body is coupled, a cover portion arranged between the trocar body and the fastening body and coupled to one side of the fastening body, and a drape portion that is arranged between the fastening body and the cover portion, and surrounds at least a portion of the robotic arm.

In some embodiments, the robotic arm may include an adapter coupling portion into which the fastening body is inserted, and a button member that is arranged in the adapter coupling portion, and separates, in response to an external input, the trocar body from the trocar adapter.

In some embodiments, the button member may include a contact portion in contact with one side of the fastening body inserted into the adapter coupling portion.

In some embodiments, in response to an external force being applied to the button member, the contact portion may cause the fastening body to be elastically deformed such that the trocar body is separated from the trocar adapter.

In some embodiments, the fastening body may include a first insertion portion that extends in one direction to be inserted into the adapter coupling portion, and comes into contact with the contact portion of the button member, and a second insertion portion extending in another direction to be inserted into the trocar body.

In some embodiments, the second insertion portion may be inserted into the trocar body through the drape portion and the cover portion.

In some embodiments, the fastening body may further include a deformation portion that connects the first insertion portion to the second insertion portion and is elastically deformable, and in response to an external force being applied to the button member, the contact portion may push the first insertion portion to cause the deformation portion to be elastically deformed, such that the second insertion portion and the trocar body are separated from each other.

In some embodiments, the trocar body may include a connection module having a trocar hole into which the second insertion portion is inserted.

In some embodiments, the robotic arm may include a fastening groove concavely formed on one side thereof and to which the fastening body is coupled.

In some embodiments, the fastening body may include a coupling portion coupled to the fastening groove of the robotic arm, and a manipulation portion that is connected to the coupling portion, and is elastically deformable.

In some embodiments, the coupling portion may include a fastening hook inserted into a recessed portion of the fastening groove.

In some embodiments, in response to an external force being applied to the manipulation portion, the manipulation portion may be elastically deformed such that the coupling portion is separated from the fastening groove.

According to another embodiment of the present disclosure, a trocar adapter includes a fastening body that connects a trocar body to a robotic arm, a cover portion arranged between the trocar body and the fastening body and coupled to one side of the fastening body, and a drape portion that is arranged between the fastening body and the cover portion, and surrounds at least a portion of the robotic arm.

In some embodiments, the fastening body may include a first insertion portion extending in one direction to be inserted into the robotic arm, and a second insertion portion extending in another direction to be inserted into the trocar body.

In some embodiments, the second insertion portion may be inserted into the trocar body through the drape portion and the cover portion.

In some embodiments, the fastening body may further include a deformation portion that connects the first insertion portion to the second insertion portion, and is elastically deformable.

In some embodiments, in response to an external force being applied to the first insertion portion, the deformation portion may be elastically deformed such that the second insertion portion and the trocar body are separated from each other.

In some embodiments, the fastening body may include a coupling portion coupled to one side of the robotic arm, and a manipulation portion that is connected to the coupling portion, and is elastically deformable.

In some embodiments, the coupling portion may include a fastening hook inserted into a recessed portion of the robotic arm.

In some embodiments, in response to an external force being applied to the manipulation portion of the fastening body, the manipulation portion may be elastically deformed such that the coupling portion is separated from the robotic arm.

Other aspects, features, advantages other than those described above will become apparent from the following drawings, claims, and detailed description of the present disclosure.

### BRIEF DESCRIPTION OF THE DRAWINGS

The above and other aspects, features, and advantages of certain embodiments of the disclosure will be more apparent from the following description taken in conjunction with the accompanying drawings in which:

FIG. 1 is a conceptual diagram illustrating a surgical robot system including a surgical robot according to an embodiment of the present disclosure;

FIG. 2 is a perspective view illustrating a partial configuration of the surgical robot of FIG. 1;

FIG. 3 is a side view of FIG. 2;

FIG. 4 is an exploded perspective view illustrating a partial configuration of the surgical robot of FIG. 2;

FIG. 5 is a diagram illustrating an interior of a robotic arm of FIG. 4;

FIGS. 6 and 7 are exploded perspective views of a trocar adapter of FIG. 4;

FIG. 8 is a diagram illustrating an example of a use state of a drape portion of FIG. 4;

FIG. 9 is a diagram illustrating an example of a state in which a trocar adapter is mounted on a robotic arm;

FIG. 10 is a diagram illustrating an example of a state in which a trocar body is mounted on a trocar adapter; and

FIG. 11 is a diagram illustrating an example of a state in which the trocar body of FIG. 10 is separated from the trocar adapter.

### DETAILED DESCRIPTION

Hereinafter, embodiments will be described in detail with reference to the accompanying drawings, and the same or corresponding components will be denoted by the same reference numerals when described with reference to the accompanying drawings, and thus their descriptions that are already provided will be omitted.

As the embodiments may be variously modified, particular embodiments will be illustrated in the drawings and described in detail in the detailed description. The effects and features of the embodiments and methods of achieving them will become clear with reference to detailed descriptions provided below with the drawings. However, the embodiments are not limited to the descriptions below, and may be implemented in various forms.

In describing the present disclosure, detailed explanations of the related art are omitted when it is deemed that they may unnecessarily obscure the gist of the present disclosure.

In the following embodiments, the singular expression also includes the plural meaning as long as it is not inconsistent with the context. In the following embodiments, terms such as "first" or "second" may be used to describe various elements, but the elements should not be limited by the terms. These terms are used only to distinguish one element from another.

In the following embodiments, the terms "comprises," "includes," "has", and the like used herein specify the presence of stated features or components, but do not preclude the presence or addition of one or more other features or components.

In the following embodiments, when a unit, region, or component is referred to as being "on" another unit, region, or component, it may be directly or indirectly on the other unit, region, or component, that is, one or more intervening units, regions, or components may be present therebetween.

In the following embodiments, when a component is referred to as being "connected to" or "coupled to" another component, the component may be directly connected to or in direct contact with the other component or intervening components may be present therebetween, unless clearly defined otherwise in the context.

For convenience of description, the magnitude of components in the drawings may be exaggerated or reduced. For example, each component in the drawings is illustrated to have an arbitrary size and thickness for ease of description, and thus the embodiments are not limited to the drawings.

In the drawings, the plane formed by the X and Y axes of the three-axis orthogonal coordinate system may be substantially parallel to a ground surface on which a surgical robot is arranged, and the Z-axis may represent the height direction of the surgical robot.

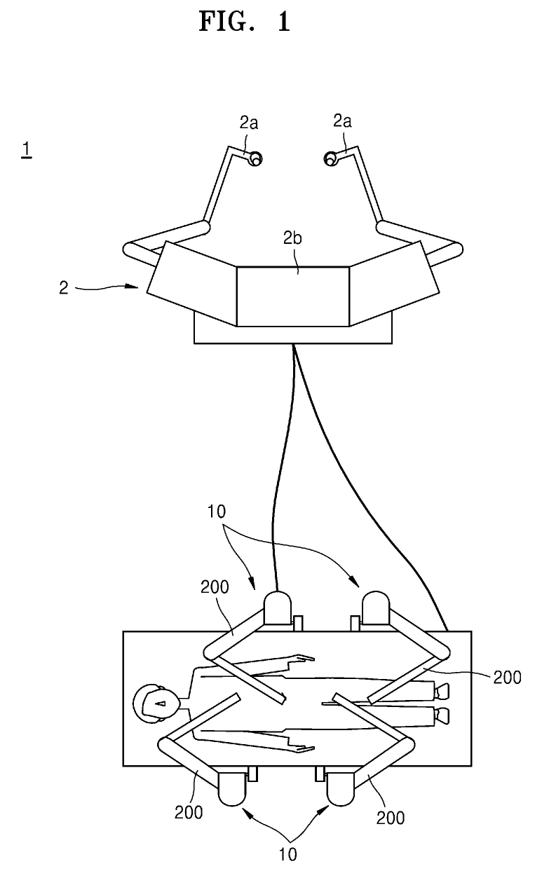

FIG. 1 is a conceptual diagram illustrating a surgical robot system 1 including a surgical robot 10 according to an embodiment of the present disclosure.

Referring to FIG. 1, the surgical robot system 1 may include a master robot 2 and the surgical robot 10.

The master robot 2 may include a manipulation member and a display member.

In detail, the master robot 2 may include a manipulation member 2a to allow an operator to hold and manipulate the manipulation member 2a with both hands. In some embodiments, an image captured through a laparoscope may be displayed as a screen image on a display member 2b of the master robot 2. In some embodiments, a predetermined virtual manipulation panel may be displayed on the display member 2b independently of or together with the image captured through the laparoscope. Detailed descriptions of the arrangement and configuration of the virtual manipulation panel will be omitted.

The surgical robot system 1 may include one or more surgical robots 10. FIG. 1 illustrates an embodiment in which the surgical robot system 1 includes four surgical robots 10.

The surgical robot 10 may include one or more robotic arms 200. As illustrated in FIG. 1, in an embodiment in which the surgical robot system 1 includes a plurality of robotic arms 200, the robotic arms 200 may be provided as modules that may operate independently of each other. Here, an algorithm for preventing collision between the robotic arms 200 may be applied to the surgical robot system 1.

In the surgical robot system 1, surgical instruments may be attached to two or more of the plurality of robotic arms 200, and laparoscopes may be attached to one or more robotic arms. In some embodiments, a surgeon may select the robotic arm 200 to be controlled via the master robot 2. As such, by the surgeon directly manipulating a total of three or more surgical instruments via the master robot 2, manipulation of various instruments may be accurately and freely performed as intended by the surgeon, without the need for a surgical assistant.

In the surgical robot system 1, a trocar body, into which a surgical instrument, a laparoscope, or the like is inserted, may be arranged in at least some of the plurality of robotic arms 200. The trocar body may be coupled to the robotic arm 200 to support a surgical instrument or the like.

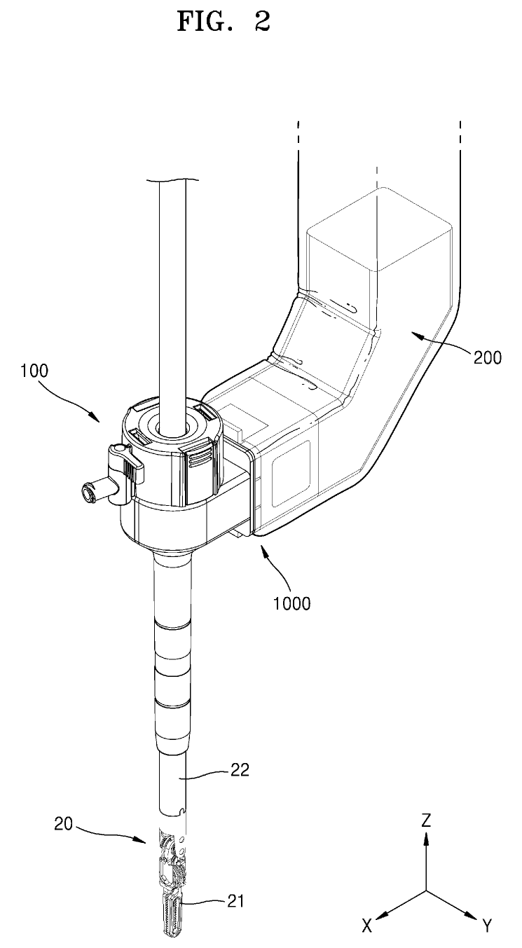

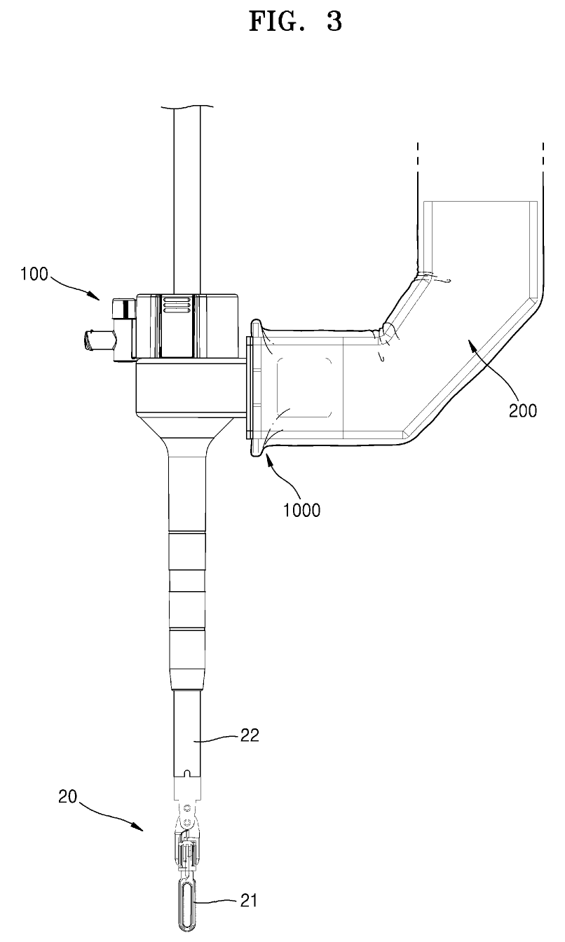

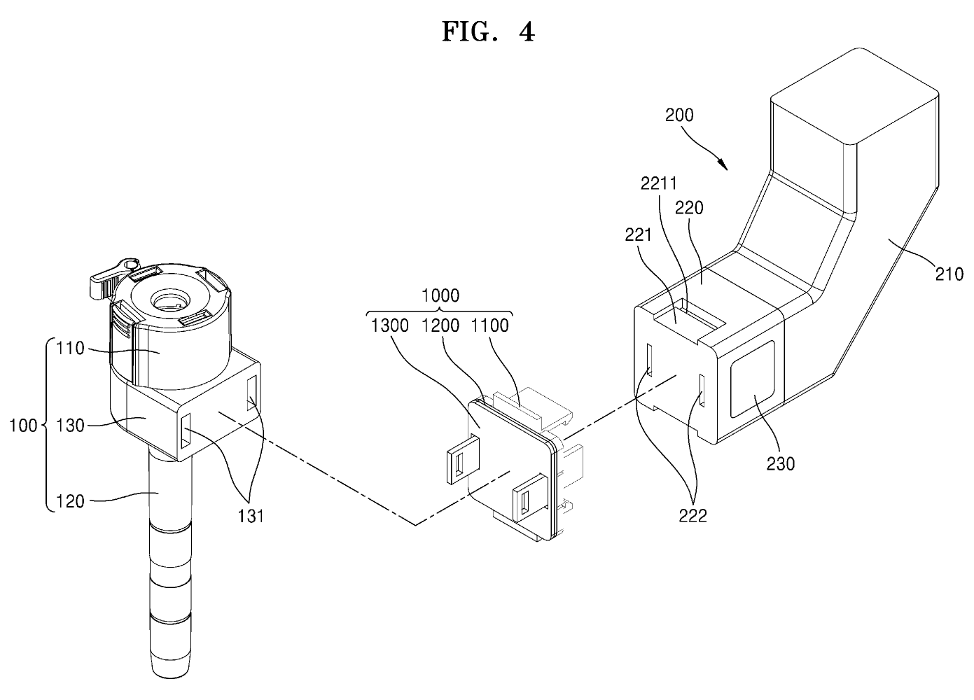

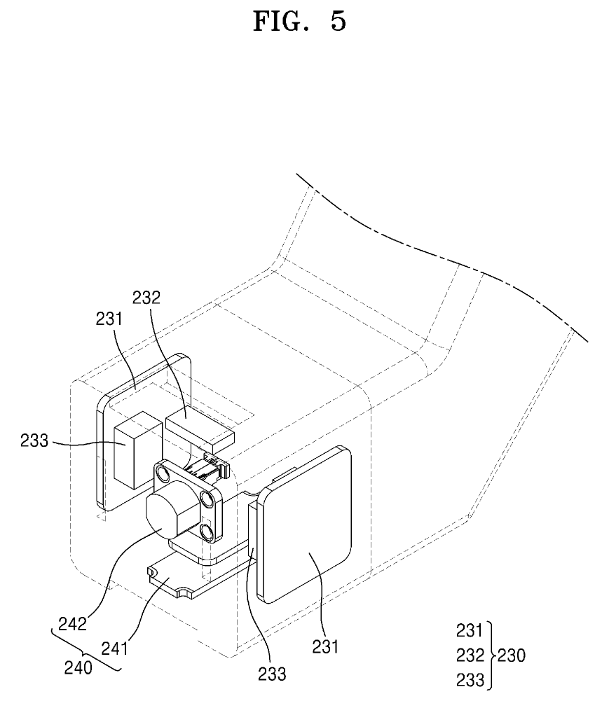

FIG. 2 is a perspective view illustrating a partial configuration of the surgical robot 10 of FIG. 1, and FIG. 3 is a side view of FIG. 2. FIG. 4 is an exploded perspective view illustrating a partial configuration of the surgical robot 10 of FIG. 2. FIG. 5 is a diagram illustrating an interior of the robotic arm 200 of FIG. 4.

Referring to FIGS. 2 and 5, the surgical robot 10 may include a trocar body 100, the robotic arm 200, and a trocar adapter 1000.

A surgical instrument 20 may be arranged in the trocar body 100. The surgical instrument 20 may be operated by manipulation by the surgical robot 10 or a user, and the trocar body 100 may support the surgical instrument 20.

The trocar body 100 may include a trocar head 110 and a trocar sleeve 120.

The surgical instrument 20 may be inserted into the trocar head 110. The trocar sleeve 120 may be connected to the trocar head 110 and may support a shaft 22 of the surgical instrument 20 inserted through the trocar head 110.

That is, the trocar head 110 and the trocar sleeve 120 may have respective internal spaces in communication with each other, and the surgical instrument 20 may be inserted into the internal spaces. With the shaft 22 of the surgical instrument 20 inserted and supported in the internal spaces, an end tool 21 of the surgical instrument 20 may perform a surgical motion.

The trocar body 100 may further include a connection module 130 to which the trocar adapter 1000 is coupled. The connection module 130 may be provided as a separate member from the trocar head 110 and the trocar sleeve 120, or may be integrally formed therewith. In some embodiments, FIG. 2 illustrates an embodiment in which the connection module 130 is arranged between the trocar head 110 and the trocar sleeve 120, but the position of the connection module 130 is not particularly limited.

The connection module 130 may include a trocar hole 131 into which the trocar adapter 1000 is inserted and fixed. In some embodiments, although not illustrated in FIG. 2 and other drawings, the connection module 130 may further include an internal locking protrusion (not shown) for coupling with the trocar adapter 1000. The method of coupling the connection module 130 to the trocar adapter 1000 will be described in detail below.

Although not illustrated in the drawings, the trocar body 100 may further include various members and structures, such as a fluid injection module for injecting fluid necessary for surgery, a manipulation module that may be directly manipulated by the user, or a display member capable of displaying surgery-related information. That is, in the present disclosure, the specific configuration, shape, and function of the trocar body 100 included in the surgical robot 10 are not particularly limited.

The robotic arm 200 may be arranged in the body of the surgical robot 10, and the surgical instrument 20, a laparoscope, or the like may be mounted on the robotic arm 200. The surgical robot 10 may drive the robotic arm 200 to adjust the position and posture of the surgical instrument 20 or the like.

The robotic arm 200 may have a link structure in which a plurality of arm links 210 are connected. FIG. 2 illustrates only one end of the robotic arm 200, that is, part of the arm link 210 farthest from the body of the surgical robot 10, and other arm links are omitted. In the present disclosure, the link structure of the robotic arm 200, and the shape, arrangement, and driving range of the arm links are not particularly limited.

The robotic arm 200 may include an adapter coupling portion 220 and a button member 230.

The trocar adapter 1000 may be coupled to the adapter coupling portion 220. The trocar adapter 1000 may be mounted on the adapter coupling portion 220, and the trocar body 100 may be inserted into the trocar adapter 1000. Accordingly, the trocar body 100 may be connected to the robotic arm 200 to perform a surgical motion.

The adapter coupling portion 220 may be arranged at one end of the arm link 210. In detail, the adapter coupling portion 220 may be arranged at the end of the arm link 210 that is farthest from the body of the surgical robot 10.

The adapter coupling portion 220 may include a fastening groove 221 and an adapter hole 222.

The fastening groove 221 may be concavely formed on one side of the adapter coupling portion 220. The fastening groove 221 may be arranged on an outer surface of the adapter coupling portion 220, and the adapter coupling portion 220 may be coupled to the fastening groove 221 such that the trocar adapter 1000 and the robotic arm 200 are fastened to each other.

The fastening groove 221 may have a recessed portion 2211. The recessed portion 2211 may be formed to be recessed from one side of the fastening groove 221, and a fastening hook 11211 of the trocar adapter 1000 may be coupled to the recessed portion 2211.

The adapter hole 222 may be formed on one surface of the adapter coupling portion 220. A portion of the trocar adapter 1000 may pass through the adapter hole 222 to be arranged inside the adapter coupling portion 220.

The button member 230 may be arranged in the adapter coupling portion 220 and may determine whether to separate the trocar adapter 1000 and the trocar body 100. The button member 230 may determine, by an external input, whether to separate the trocar adapter 1000 inserted into the trocar body 100.

In an embodiment, a pair of button members 230 may be provided. In FIG. 4, a pair of button members 230 may be arranged on opposite side surfaces of the adapter coupling portion 220.

The button member 230 may include a pad portion 231, a support portion 232, and a contact portion 233.

The pad portion 231 may be arranged on an outer surface of the adapter coupling portion 220 and may be provided to be movable by a predetermined amount. In detail, the pad portion 231 may be pushed, by an external input, toward the inside of the adapter coupling portion 220 by a predetermined distance. Here, the external input may be a manual manipulation by the user or a control signal transmitted from the surgical robot system 1 in a wired or wireless manner.

The support portion 232 may extend from the pad portion 231 and may be arranged inside the adapter coupling portion 220.

In an embodiment, the support portion 232 is elastically deformable. When an external force is applied to the pad portion 231, the support portion 232 may be elastically compressed such that the pad portion 231 moves toward the inside of the adapter coupling portion 220 by a predetermined distance. When the external force applied to the pad portion 231 is removed, the support portion 232 may return to its original state, and the pad portion 231 may also return to its initial position.

The contact portion 233 may come into contact with the trocar adapter 1000 inserted into the adapter coupling portion 220. The contact portion 233 may apply an external force to one side of the trocar adapter 1000, and as a result, the trocar adapter 1000 may be separated from the adapter coupling portion 220.

The specific method by which the trocar adapter 1000 is separated by the button member 230 will be described in detail below.

The robotic arm 200 may further include a sensor module 240. The sensor module 240 may sense the trocar body 100 mounted on the robotic arm 200.

The sensor module 240 may include a sensor unit 241 coupled to the trocar adapter 1000 and configured to identify the trocar body 100 mounted on the robotic arm 200, and a communication unit 242 connected to the sensor unit 241 and configured to transmit and receive information about the trocar body 100.

Because the trocar body 100 is mounted on the robotic arm 200 via the trocar adapter 1000, the robotic arm 200 may identify information about the trocar body 100 through the sensor module 240 and use the information to perform a surgical motion.

The trocar adapter 1000 may connect the trocar body 100 to the robotic arm 200. The trocar adapter 1000 may be arranged between the trocar body 100 and the robotic arm 200, with one side coupled to the robotic arm 200 and another side coupled to the trocar body 100.

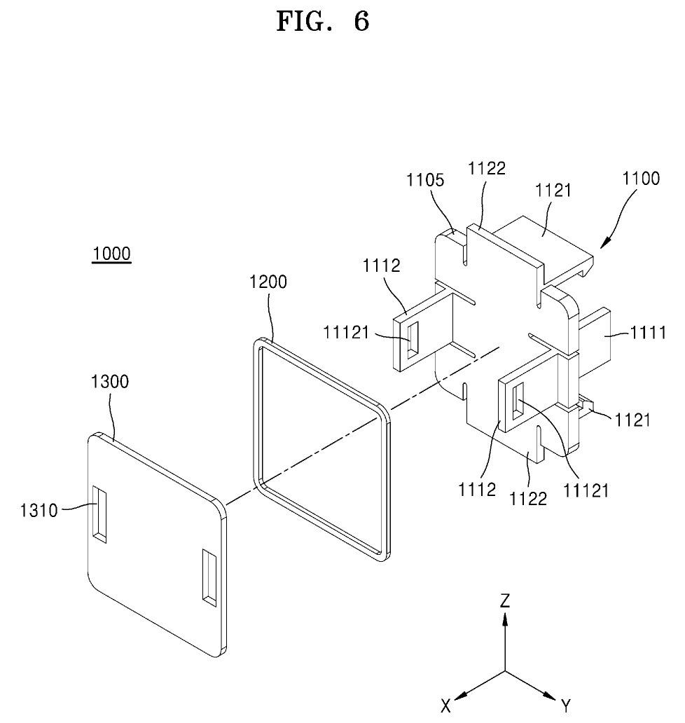

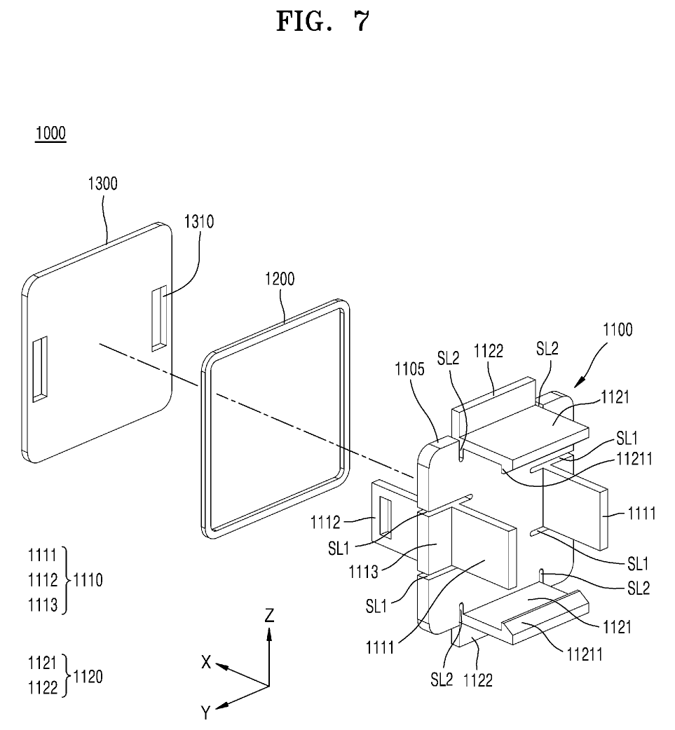

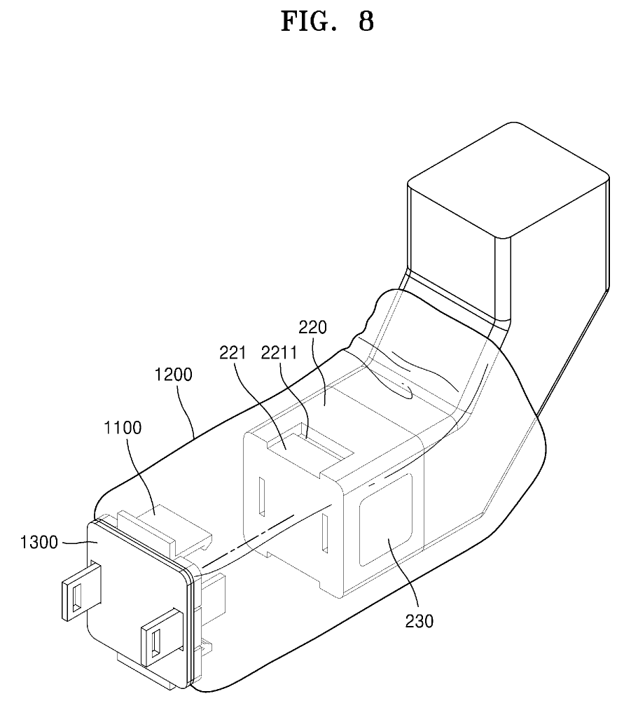

FIGS. 6 and 7 are exploded perspective views of the trocar adapter 1000 of FIG. 4, and FIG. 8 is a diagram illustrating an example of a use state of a drape portion 1200 of FIG. 4.

Referring to FIGS. 6 and 8, the trocar adapter 1000 may include a fastening body 1100, the drape portion 1200, and a cover portion 1300.

The fastening body 1100 may be mounted on the robotic arm 200, and the trocar body 100 may be coupled to the fastening body 1100.

The fastening body 1100 may include a base plate 1105, a first fastening portion 1110, and a second fastening portion 1120.

The base plate 1105 may have a substantially plate shape, and the first fastening portion 1110 may be formed by cutting out a partial area of the base plate 1105.

In detail, the first fastening portion 1110 may be arranged between a pair of first slits SL1 cut toward the center from one side surface of the base plate 1105. Similarly, the second fastening portion 1120 may be arranged between a pair of second slits SL2 cut toward the center from another side surface of the base plate 1105. Accordingly, the first fastening portion 1110 and the second fastening portion 1120 may be connected to the base plate 1105 and may be provided to be elastically deformable.

A pair of first fastening portions 1110 and a pair of second fastening portions 1120 may be provided. The pair of the first fastening portions 1110 may be arranged on one side surface of the base plate 1105 and on the opposite side surface, respectively. Similarly, the pair of the second fastening portions 1120 may be arranged on another side surface of the base plate 1105 and on the opposite side surface, respectively.

The first fastening portion 1110 may be coupled to the robotic arm 200 and the trocar body 100. In some embodiments, in a state in which the trocar body 100 is coupled to the trocar adapter 1000, the first fastening portion 1110 may be elastically deformed by the button member 230 such that the trocar body 100 is separated from the trocar adapter 1000.

The first fastening portion 1110 may include a first insertion portion 1111, a second insertion portion 1112, and a deformation portion 1113.

The first insertion portion 1111 may extend in one direction to be inserted into the adapter coupling portion 220.

The first insertion portion 1111 may come into contact with the button member 230. The first insertion portion 1111 may come into contact with the contact portion 233 of the button member 230 and may receive an external force from the contact portion 233.

The second insertion portion 1112 may extend in another direction to be inserted into the trocar body 100. The second insertion portion 1112 may extend in the opposite direction to the first insertion portion 1111 to be inserted into the trocar hole 131 of the trocar body 100. Here, the second insertion portion 1112 may pass through the drape portion 1200 and the cover portion 1300 to be inserted into the trocar body 100.

In an embodiment, the second insertion portion 1112 may have a locking hole 11121. When the second insertion portion 1112 is inserted into the trocar body 100, the locking hole 11121 may fasten the trocar body 100 to the trocar adapter 1000. Depending on the position of the second insertion portion 1112, which is determined by the elastic deformation of the deformation portion 1113, the locking hole 11121 may be coupled to or released from a locking protrusion (not shown) provided inside the trocar body 100. Accordingly, the trocar body 100 and the trocar adapter 1000 may be coupled to or separated from each other.

The deformation portion 1113 may connect the first insertion portion 1111 to the second insertion portion 1112 and may be elastically deformed. A partial area of the deformation portion 1113 may be formed to be separated from the base plate 1105 by the first slits SL1 and then elastically deformed. When an external force is applied to the first insertion portion 1111, the deformation portion 1113 may be elastically deformed to change the positions of the first insertion portion 1111 and the second insertion portion 1112. Accordingly, the second insertion portion 1112 and the trocar body 100 may be coupled to or separated from each other.

The second fastening portion 1120 may include a coupling portion 1121 and a manipulation portion 1122.

The coupling portion 1121 may be coupled to the fastening groove 221 of the robotic arm 200. The coupling portion 1121 may extend in one direction, which is identical to the extension direction of the first insertion portion 1111, to be coupled to the fastening groove 221.

The coupling portion 1121 may have the fastening hook 11211. The fastening hook 11211 may protrude at a predetermined angle with respect to the extension direction of the coupling portion 1121. When the coupling portion 1121 is coupled to the fastening groove 221, the fastening hook 11211 may be inserted into the recessed portion 2211 of the fastening groove 221, such that the trocar adapter 1000 is more stably fastened to the robotic arm 200.

The manipulation portion 1122 may be connected to the coupling portion 1121 and may be elastically deformed. A partial area of the manipulation portion 1122 may be provided to be separated from the base plate 1105 by the second slits SL2 and then elastically deformed. When an external force is applied to the manipulation portion 1122, the manipulation portion 1122 may be elastically deformed to cause the coupling portion 1121 to be separated from the fastening groove 221.

The drape portion 1200 may be coupled to the fastening body 1100 and may surround at least a portion of the robotic arm 200.

The drape portion 1200 may include various materials such as polypropylene, polyethylene, or acrylic, and may be made of any material capable of blocking bacteria and other foreign substances. As the drape portion 1200 covers at least a portion of the robotic arm 200, the trocar body 100 may be connected to the robotic arm 200 while the robotic arm 200 is kept sterile.

FIGS. 6 and 7 illustrate only a portion of the drape portion 1200 that is coupled to the fastening body 1100, but the drape portion 1200 may extend in the extension direction of the robotic arm 200 to surround the robotic arm 200, as illustrated in FIG. 8.

The cover portion 1300 may cover one side of the fastening body 1100. The cover portion 1300 may be coupled to the fastening body 1100, positioned between the trocar body 100 mounted on the trocar adapter 1000 and the fastening body 1100.

The cover portion 1300 may have a shape substantially corresponding to the base plate 1105 of the fastening body 1100. The cover portion 1300 may include a through hole 1310, and the second insertion portion 1112 of the fastening body 1100 may pass through the through hole 1310 to be inserted into the trocar body 100.

In summary, the fastening body 1100 may be fastened to the adapter coupling portion 220 of the robotic arm 200, and the trocar body 100 may be mounted on the fastening body 1100. Accordingly, the trocar body 100 may be connected to the robotic arm 200 via the trocar adapter 1000.

In some embodiments, a portion of the drape portion 1200 may be arranged and fixed between the fastening body 1100 and the cover portion 1300. The drape portion 1200 may extend from between the fastening body 1100 and the cover portion 1300 to surround at least a portion of the robotic arm 200. Accordingly, the trocar body 100 may be easily attached to and detached from the robotic arm 200 while the robotic arm 200 is kept sterile, and may seamlessly operate.

Hereinafter, coupling and separation processes of the trocar body 100, the trocar adapter 1000, and the robotic arm 200 will be described in detail.

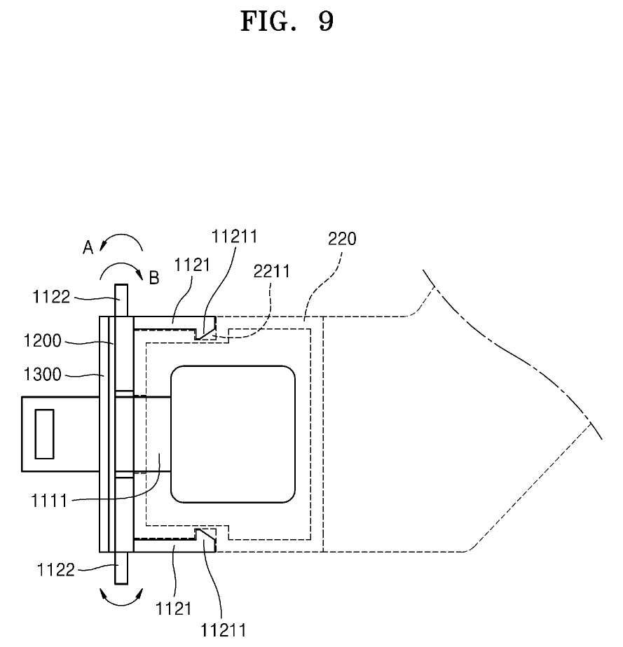

FIG. 9 is a diagram illustrating an example of a state in which the trocar adapter 1000 is mounted on the robotic arm 200.

Referring to FIG. 9, a portion of the first fastening portion 1110 of the trocar adapter 1000 may be inserted into the adapter coupling portion 220. In some embodiments, the second fastening portion 1120 of the trocar adapter 1000 may be coupled to an outer surface of the adapter coupling portion 220.

In detail, the first insertion portion 1111 of the first fastening portion 1110 may be inserted into the adapter coupling portion 220. The first insertion portion 1111 may pass through the adapter hole 222 of the adapter coupling portion 220 to be arranged inside the robotic arm 200.

In some embodiments, the coupling portion 1121 of the second fastening portion 1120 may be coupled to the fastening groove 221, at an outer portion of the adapter coupling portion 220. The fastening hook 11211 of the coupling portion 1121 may be inserted into the recessed portion 2211 of the fastening groove 221. Accordingly, the trocar adapter 1000 may be stably mounted on the robotic arm 200.

The manipulation portion 1122 of the second fastening portion 1120 may be elastically deformed by an external input, and the trocar adapter 1000 may be coupled to or separated from the robotic arm 200 according to the elastic deformation of the manipulation portion 1122.

In an embodiment, in a state in which a certain external force is applied to the manipulation portion 1122, the trocar adapter 1000 may be coupled to the adapter coupling portion 220. The manipulation portion 1122 of the second fastening portion 1120 may be provided to be elastically deformable and thus may be elastically deformed by an external input such as an external force. For example, when an external force in direction A is applied to the manipulation portion 1122, the manipulation portion 1122 and the coupling portion 1121 may rotate together by a certain angle in direction A. Thereafter, when the external force applied to the manipulation portion 1122 is removed, the manipulation portion 1122 may return to its initial state by a restoring force in direction B. Accordingly, the coupling portion 1121 may be inserted into the fastening groove 221 such that the trocar adapter 1000 is fastened to the robotic arm 200.

In some embodiments, in a state in which the trocar adapter 1000 is coupled and fixed to the robotic arm 200, when an external force in direction A is applied to the manipulation portion 1122, the manipulation portion 1122 and the coupling portion 1121 may rotate together by a certain angle in direction A. Accordingly, the coupling of the coupling portion 1121 and the fastening groove 221 may be released, and the trocar adapter 1000 may be separated from the robotic arm 200.

As such, the trocar adapter 1000 may be quickly and easily coupled to or separated from the robotic arm 200 simply by manipulating the elastically deformable manipulation portion 1122. Here, the user may manually apply an external force to the manipulation portion 1122, or the manipulation portion 1122 may be automatically manipulated by an external control signal or the like. In some embodiments, in an embodiment in which the trocar adapter 1000 includes a pair of second fastening portions 1120 as illustrated in FIG. 9, a pair of manipulation portions 1122 may be manipulated simultaneously or independently.

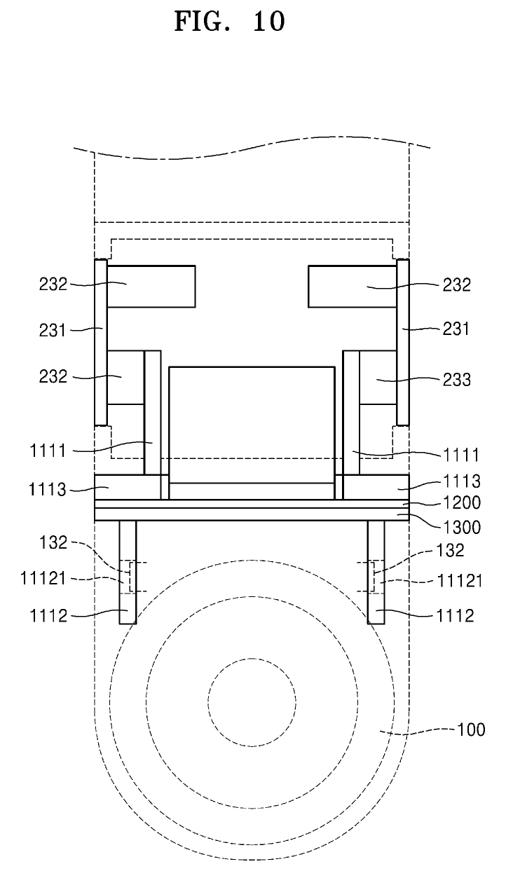

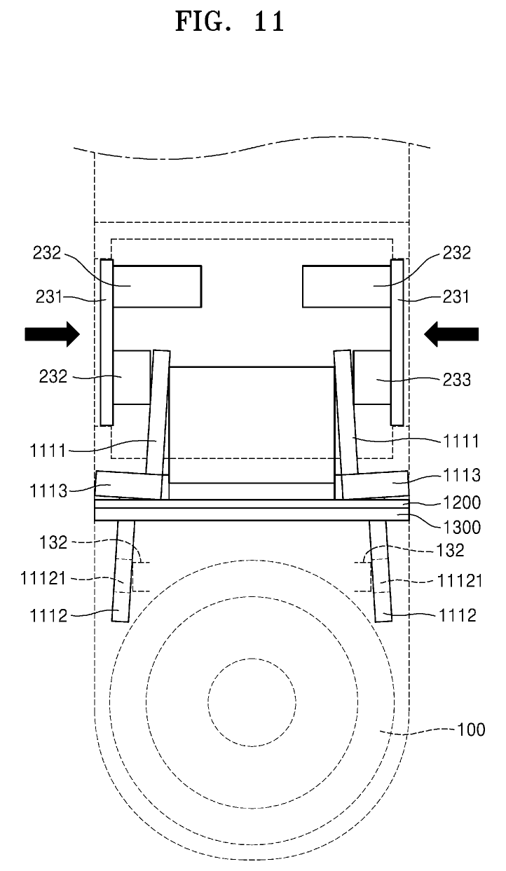

FIG. 10 is a diagram illustrating an example of a state in which the trocar body 100 is mounted on the trocar adapter 1000, and FIG. 11 is a diagram illustrating an example of a state in which the trocar body 100 of FIG. 10 is separated from the trocar adapter 1000.

Referring to FIGS. 10 and 11, a portion of the first fastening portion 1110 of the trocar adapter 1000 may be inserted into the trocar body 100.

In an embodiment, the second insertion portion 1112 of the first fastening portion 1110 may engage with a locking protrusion 132 of the trocar body 100. When the second insertion portion 1112 is inserted into the trocar hole 131 of the trocar body 100, the locking protrusion 132 may come into contact with the second insertion portion 1112 to cause the deformation portion 1113 to be elastically deformed, and thus, the position of the second insertion portion 1112 may change. In other words, the second insertion portion 1112 may be pushed by a certain amount toward an edge of the trocar body 100 in the drawing.

In this process, when the locking protrusion 132 is inserted into the locking hole 11121 of the second insertion portion 1112, the second insertion portion 1112 may return to its initial position by a restoring force. Accordingly, the trocar body 100 may be stably coupled and fixed to the trocar adapter 1000.

However, the method of fastening the trocar adapter 1000 to the trocar body 100 is not particularly limited, and any method may be selected as long as the second insertion portion 1112 may be inserted and fixed into the trocar body 100.

In some embodiments, in a state in which the trocar adapter 1000 mounted on the robotic arm 200, the first insertion portion 1111 of the first fastening portion 1110 may come into contact with the contact portion 233 of the button member 230.

The button member 230 may be provided to be movable by a certain amount toward the inside of the adapter coupling portion 220 by an external input. A force may be applied to the first insertion portion 1111 by a movement of the button member 230, and the deformation portion 1113 connected to the first insertion portion 1111 may be elastically deformed such that the position of the second insertion portion 1112 changes.

In other words, when the contact portion 233 of the button member 230 pushes the first insertion portion 1111 toward the center of the adapter coupling portion 220, the second insertion portion 1112 may move by a certain amount toward an edge of the adapter coupling portion 220 due to the deformation of the deformation portion 1113. Accordingly, the coupling between the locking hole 11121 of the second insertion portion 1112 and the locking protrusion 132 may be released, and the trocar body 100 may be separated from the trocar adapter 1000.

As such, the trocar body 100 may be quickly and easily separated from the trocar adapter 1000 by manipulating the button member 230. Here, the button member 230 may be manually manipulated by the user's pressing motion or the like, or may be automatically manipulated in response to receiving a control signal from the surgical robot system 1 in a wired or wireless manner. In some embodiments, in an embodiment in which a pair of button members 230 are provided as illustrated in FIGS. 10 and 11, the pair of button members 230 may be manipulated simultaneously or independently.

In summary, the trocar adapter 1000 may be mounted on the robotic arm 200 according to a manipulation of the second fastening portion 1120, specifically, the manipulation portion 1122. Here, the first insertion portion 1111 of the trocar adapter 1000 may be inserted into the robotic arm 200. In a state in which the fastening body 1100 of the trocar adapter 1000 is fastened to the robotic arm 200, the drape portion 1200 may surround at least a portion of the robotic arm 200 to keep the robotic arm 200 sterile.

The trocar body 100 may be coupled and fixed to the second insertion portion 1112 of the trocar adapter 1000. In this state, the surgical instrument 20 or the like that is inserted into the trocar body 100 may perform a surgical motion. In some embodiments, the trocar body 100 may be separated from the trocar adapter 1000 according to a manipulation of the button member 230. In some embodiments, the trocar adapter 1000 may be separated from the robotic arm 200 according to a manipulation of the second fastening portion 1120, specifically, the manipulation portion 1122.

In a trocar adapter and a surgical robot including the same according to the present disclosure, a robotic arm and a trocar body may be connected to each other, and a drape portion may surround at least a portion of the robotic arm. Accordingly, the trocar body may be easily mounted on the robotic arm while the robotic arm is kept sterile.

In the trocar adapter and the surgical robot including the same according to the present disclosure, a partial area of a fastening body connecting the robotic arm to the trocar body is provided to be elastically deformable, such that coupling or separation between the trocar body, the trocar adapter, and the robotic arm may be easily and quickly performed by a manipulation of a manipulation portion, a button member of the robotic arm, or the like.

The present disclosure has been described with reference to the preferred embodiments. It will be understood by those of skill in the art that the present disclosure may be implemented in a modified form without departing from the intrinsic characteristics of the present disclosure. Therefore, the disclosed embodiments are to be considered in a descriptive sense only, and not for purposes of limitation. The scope of the present disclosure is in the claims rather than the above descriptions, and all differences within the equivalent scope should be construed as being included in the present disclosure.

In a trocar adapter and a surgical robot including the same according to an embodiment of the present disclosure, a trocar body and a robotic arm may be connected to each other, and a drape portion may surround the robotic arm. In the trocar adapter and the surgical robot including the same according to an embodiment of the present disclosure, the trocar body is mounted while the drape portion covers the robotic arm, such that the trocar body may seamlessly operate while the sterility of the robotic arm is maintained.

What is claimed is:

1. A surgical robot comprising:
a trocar body into which a surgical instrument is inserted;
a robotic arm in which the trocar body is arranged; and
a trocar adapter that connects the trocar body to the robotic arm,
wherein the trocar adapter comprises:
a fastening body mounted on the robotic arm and to which the trocar body is coupled;
a cover portion arranged between the trocar body and the fastening body and coupled to one side of the fastening body; and
a drape portion that is arranged between the fastening body and the cover portion, and surrounds at least a portion of the robotic arm.

2. The surgical robot of claim 1, wherein the robotic arm comprises:
an adapter coupling portion into which the fastening body is inserted; and
a button member that is arranged in the adapter coupling portion, and separates, in response to an external input, the trocar body from the trocar adapter.

3. The surgical robot of claim 2, wherein the button member comprises a contact portion in contact with one side of the fastening body inserted into the adapter coupling portion.

4. The surgical robot of claim 3, wherein, in response to an external force being applied to the button member, the contact portion causes the fastening body to be elastically deformed such that the trocar body is separated from the trocar adapter.

5. The surgical robot of claim 3, wherein the fastening body comprises:
a first insertion portion that extends in one direction to be inserted into the adapter coupling portion, and comes into contact with the contact portion of the button member; and
a second insertion portion extending in another direction to be inserted into the trocar body.

6. The surgical robot of claim 5, wherein the second insertion portion is inserted into the trocar body through the drape portion and the cover portion.

7. The surgical robot of claim 5, wherein the fastening body further comprises a deformation portion that connects the first insertion portion to the second insertion portion and is elastically deformable, and
in response to an external force being applied to the button member, the contact portion pushes the first insertion portion to cause the deformation portion to be elastically deformed, such that the second insertion portion and the trocar body are separated from each other.

8. The surgical robot of claim 5, wherein the trocar body comprises a connection module having a trocar hole into which the second insertion portion is inserted.

9. The surgical robot of claim 1, wherein the robotic arm comprises a fastening groove concavely formed on one side thereof and to which the fastening body is coupled.

10. The surgical robot of claim 9, wherein the fastening body comprises:
a coupling portion coupled to the fastening groove of the robotic arm; and
a manipulation portion that is connected to the coupling portion, and is elastically deformable.

11. The surgical robot of claim 10, wherein the coupling portion comprises a fastening hook inserted into a recessed portion of the fastening groove.

12. The surgical robot of claim 10, wherein, in response to an external force being applied to the manipulation portion, the manipulation portion is elastically deformed such that the coupling portion is separated from the fastening groove.

13. A trocar adapter comprising:
a fastening body that connects a trocar body to a robotic arm;
a cover portion arranged between the trocar body and the fastening body and coupled to one side of the fastening body; and
a drape portion that is arranged between the fastening body and the cover portion, and surrounds at least a portion of the robotic arm.

14. The trocar adapter of claim 13, wherein the fastening body comprises:
a first insertion portion extending in one direction to be inserted into the robotic arm; and
a second insertion portion extending in another direction to be inserted into the trocar body.

15. The trocar adapter of claim 14, wherein the second insertion portion is inserted into the trocar body through the drape portion and the cover portion.

16. The trocar adapter of claim 14, wherein the fastening body further comprises:
a deformation portion that connects the first insertion portion to the second insertion portion, and is elastically deformable.

17. The trocar adapter of claim 16, wherein, in response to an external force being applied to the first insertion portion, the deformation portion is elastically deformed such that the second insertion portion and the trocar body are separated from each other.

18. The trocar adapter of claim 13, wherein the fastening body comprises:
a coupling portion coupled to one side of the robotic arm; and
a manipulation portion that is connected to the coupling portion, and is elastically deformable.

19. The trocar adapter of claim 18, wherein the coupling portion comprises a fastening hook inserted into a recessed portion of the robotic arm.

20. The trocar adapter of claim 18, wherein, in response to an external force being applied to the manipulation portion of the fastening body, the manipulation portion is elastically deformed such that the coupling portion is separated from the robotic arm.
